(19) United States
(12) Patent Application Publication
(10) Pub. No.: US 2026/0134943 A1
(43) Pub. Date: May 14, 2026

(54) USE OF RAD51 MUTATIONS AS A PREDICTIVE BIOMARKER FOR USE OF PARP INHIBITOR IN CANCER TREATMENT

(71) Applicant: Foundation Medicine, Inc., Cambridge, MA (US)

(72) Inventors: Ryon P. Graf, Encinitas, CA (US); Julia A. Elvin, Newtonville, MA (US)

(73) Assignee: Foundation Medicine, Inc., Cambridge, MA (US)

(21) Appl. No.: 19/389,011

(22) Filed: Nov. 13, 2025

Related U.S. Application Data
(60) Provisional application No. 63/720,579, filed on Nov. 14, 2024.

(51) Int. Cl. G16B 20/20; C12Q 1/6855; C12Q 1/6874; G16B 30/00; G16B 40/20; G16H 15/00; G16H 20/17; G16H 50/20

(52) U.S. Cl. CPC G16B 20/20; C12Q 1/6855; C12Q 1/6874; G16B 30/00; G16B 40/20; G16H 15/00; G16H 20/17; G16H 50/20; C12Q 2600/106; C12Q 2600/158

(57) ABSTRACT

Methods are described for identifying and/or treating patients who may benefit from PARP inhibitors based on an HRDsig status and a RAD51 gene status identified using genomic profiling data.

### CROSS-REFERENCE TO RELATED APPLICATION

This application claims priority to U.S. Provisional App. No. 63/720,579, which was filed on Nov. 14, 2024 and is incorporated by reference herein in its entirety.

### FIELD

The present disclosure relates generally to methods for predicting the use of PARP inhibitors in cancer treatment, and more specifically to methods for identifying and/or treating patients who may benefit from PARP inhibitors immunotherapy based on HRDsig status and a RAD51 gene status.

### BACKGROUND

PARP inhibitors are a major component of the standard of care of patients with advanced ovarian cancer. However, these drugs have been shown to confer the most benefit to PARPi are in patients whose tumor cells have deficiencies in homologous recombination repair (HRR) pathway. Mechanisms of HRR pathway perturbation include epigenomic silencing of promoter regions, bi-allelic deleterious mutations, hemizygous loss of gene and one deleterious mutation, and homozygous loss of the gene which can also be referred to as a “loss” or “homozygous deletion.”

There have been reports of BRCA2 loss and RAD51C and RAD51D mutations being associated with durable (1 year or greater) benefit from recurrence-setting PARPi. Prior post-hoc studies of ARIEL2 also evaluated BRCA alterations by alteration class, combining homozygous loss and rearrangements together and reporting more durable benefit from PARPi in this group. A recent study evaluating routine practice use of PARPi in the treatment of advanced prostate cancer observed the most durable benefit from PARPi associated with homozygous loss of the HRR genes BRCA1 and BRCA2. There is an existing body of biological evidence to suggest that a common mechanism of resistance to PARPi is the evolution of reversion mutations and that complete loss of HRR gene(s) may confer the inability to evolve this resistance mechanism.

### BRIEF SUMMARY OF THE INVENTION

At present, PARPi monotherapy is predominantly used in the maintenance setting for patients with a partial or complete response to platinum-containing primary chemotherapy, with addition of bevacizumab if also used during primary therapy. While the National Comprehensive Cancer Network (NCCN) guidelines acknowledges BRCA1/2 mutation status within the maintenance therapy decision tree, the weight that this information should be given in selecting a PARPi maintenance regimen versus observation or bevacizumab alone is unclear. It has previously been shown that the HRD signature (HRDsig) status was associated with progression-free survival (PFS) benefit of PARPi maintenance6 in the platinum-sensitive population, as well as predicting lack of benefit from PARPi for those HRDsig(−). The current study further elaborates the study of this cohort by other factors, such as HRR homozygous losses and other HRR alterations to provide further mechanisms for risk stratification of patients receiving PARPi and to identify those patients likely to receive the greatest benefit.

Disclosed herein are methods of treating a subject having ovarian cancer, the method comprising: acquiring a genomic profile based on a sample from the subject, wherein the genomic profile is indicative of: (i) an HRDsig positive status, and (ii) the presence of a RAD51C loss or a RAD51D loss; and responsive to an indication that an HRD positive status is present with the presence of at least one of a RAD51C loss or a RAD51D loss, administering an anti-cancer therapy to the subject, thereby treating the subject with the anti-cancer therapy.

In some embodiments the anti-cancer therapy comprises a PARP inhibitor.

Disclosed herein are methods for identifying a cancer patient as a candidate for treatment with a PARP inhibitor, the method comprising: receiving, at one or more processors, genomic data derived from a sample from the subject; providing, using the one or more processors, the genomic data as input to a first trained model configured to identify a HRDsig status; providing, using the one or more processors, the genomic data as input to a second trained machine model configured to identify a RAD51C status and/or a RAD51D status; and identifying the cancer patient as a candidate for treatment with the PARP inhibitor based on: (i) an output from the first trained model is indicative that the HRDsig status is positive, and (ii) an output from the second trained model is indicative of a RAD51C loss and/or a RAD51D loss.

In some embodiments, the first trained model and the second trained model are a singular model.

In some embodiments, the cancer comprises an ovarian cancer.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIGS. 1A-1C provide non-limiting examples of cohort selection. Temporal visualization of the cohorts is shown for patients receiving PARPi in the 2nd+ line setting after recurrence from platinum therapy (FIG. 1B) and illustrates for maintenance therapy cohort after 1st line chemotherapy (FIG. 1C).

FIG. 2 provides a non-limiting example of data that illustrates prevalence of each alteration class in genes of the HRR pathway.

FIGS. 3A-3C provide non-limiting examples of data that illustrate outcomes on PARPi monotherapy associated with specific alteration type are visualized with Kaplan-Meier plots for PFS (FIG. 3A) and OS (FIG. 3B). Swim lane plot (FIG. 3C) visualizes specific alterations.

FIGS. 4A-4C provide non-limiting examples of data that illustrates Forest plots to visualize outcomes on PARPi maintenance therapy associated with alteration types (FIG. 4A) and additionally by HRDsig status and BRCA status (FIG. 4B). Swim lane plot (FIG. 4C) visualizes specific alterations. Vertical dashed line indicates the median PFS for patients with no maintenance therapy.

FIG. 5 provides a non-limiting example of a multivariable assessment of PFS on 2nd+ line PARPi monotherapy by alteration class. The independent associations of HRR alteration class with progression-free survival on PARPi in the recurrence setting was adjusted for potential imbalances in prognostic factors or factors that might associate with PARPi sensitivity.

FIGS. 6A and 6B provide non-limiting examples of data that illustrate Cohort Selection for Platinum Therapy Cohort. Cohort selection diagram (FIG. 6A) and temporal visualization of the cohort (FIG. 6B) are shown for patients receiving 1st line platinum therapy without PARPi maintenance therapy.

FIG. 7 provides non-limiting examples of data for multivariable assessment of PFS on 1st line platinum therapy by alteration class. The independent associations of HRR alteration class with progression-free survival on PARPi in the recurrence setting was adjusted for potential imbalances in prognostic factors.

### EXAMPLES

### I. Abstract_

While poly ADP-ribose polymerase inhibitors (PARPi) are used ubiquitously in the care of patients with advanced ovarian cancer, the durability of clinical benefit is variable. The most established mechanism of resistance to PARPi is the development of reversion mutations that restore homologous recombination repair (HRR). Further characterization of the clinical associations of PARPi use in patients whose tumors contain homozygous loss of HRR pathway genes that are incapable of forming reversion mutations that underlie acquired PARPi resistance was performed.

### II. Methods

Patient-level data from a deidentified nationwide (U.S.-based) cancer clinico-genomic database between January 2011 and March 2024 were extracted. Patients with advanced ovarian cancer and comprehensive genomic profiling by CGP were assembled into cohorts evaluating outcomes associated with PARPi monotherapy or PARPi maintenance therapy.

### III. Results

The prevalence of HRR gene homozygous loss was 1.6% (88/5536), with BRCA1 and BRCA2 loss being most common. Maintenance therapy cohort: 21 patients had HRR gene homozygous loss, with strongly more favorable PFS for PARPi use (HR: 0.10, 95% CI: 0.02-0.46). PARPi monotherapy cohort: patients with no HRR alterations (n=183), HRR mutations or rearrangements (n=100), and HRR homozygous loss (n=7) had median PFS of 4.9, 10.6 and 25.4 months, and median OS of 18.5, 22.5, and 59.4 months, respectively.

### IV. Conclusions

Durable PARPi effectiveness for patients with HRR gene homozygous loss in routine practice was observed. The importance of detecting and differentiating homozygous loss in HRR genes, an aspect not standard to all sequencing assays was demonstrated.

### V. Introduction

PARP inhibitors are a major component of the standard of care of patients with advanced ovarian cancer. However, these drugs have been shown to confer the most benefit to PARPi are in patients whose tumor cells have deficiencies in homologous recombination repair (HRR) pathway. Mechanisms of HRR pathway perturbation include epigenomic silencing of promoter regions, bi-allelic deleterious mutations, hemizygous loss of gene and one deleterious mutation, and homozygous loss of the gene which can also be referred to as a “loss” or “homozygous deletion” (REF).

There have been reports of BRCA2 loss and RAD51C and RAD51D mutations being associated with durable (1 year or greater) benefit from recurrence-setting PARPi. Prior post-hoc studies of ARIEL2 also evaluated BRCA alterations by alteration class, combining homozygous loss and rearrangements together and reporting more durable benefit from PARPi in this group2. A recent study evaluating routine practice use of PARPi in the treatment of advanced prostate cancer observed the most durable benefit from PARPi associated with homozygous loss of the HRR genes BRCA1 and BRCA23. There is an existing body of biological evidence to suggest that a common mechanism of resistance to PARPi is the evolution of reversion mutations and that complete loss of HRR gene(s) may confer the inability to evolve this resistance mechanism.

At present, PARPi monotherapy is predominantly used in the maintenance setting for patients with a partial or complete response to platinum-containing primary chemotherapy, with addition of bevacizumab if also used during primary therapy. While the National Comprehensive Cancer Network (NCCN) guidelines acknowledges BRCA1/2 mutation status within the maintenance therapy decision tree, the weight that this information should be given in selecting a PARPi maintenance regimen versus observation or bevacizumab alone is unclear. It has been shown that the HRD signature (HRDsig) status was associated with progression-free survival (PFS) benefit of PARPi maintenance in the platinum-sensitive population, as well as predicting lack of benefit from PARPi for those HRDsig(−). The current study further elaborates the study of this cohort by other factors, such as HRR homozygous losses and other HRR alterations to provide further mechanisms for risk stratification of patients receiving PARPi and to identify those patients likely to receive the greatest benefit.

### VI. Methods

VI.a. Study Design and Patient Selection

The cohort consisted of patients with confirmed diagnosis of ovarian cancer included in a de-identified clinico-genomic database (CGDB) between January 2011-June 2024. All patients underwent genomic testing using comprehensive genomic profiling (CGP) assays (described below) with molecular data linked directly.

De-identified clinical data originated from approximately 280 US cancer clinics (˜800 sites of care). Retrospective longitudinal clinical data were derived from electronic health records (EHR), comprising patient-level structured and unstructured data, curated via technology-enabled abstraction of clinical notes and radiology/pathology reports, which were linked to genomic data derived from CGP testing by de-identified, deterministic matching. Clinical data included demographics, clinical and laboratory features, timing of treatment exposure, treatment progression, survival, and orthogonal unstructured somatic and germline molecular testing abstracted from PDF reports of different CGP testing. Lines of therapy in the database were oncology clinician-defined and rule-based. Patients with a Birth Year of 1938 or earlier may have an adjusted Birth Year in datasets due to patient de-identification requirements.

The study population comprises three cohorts:

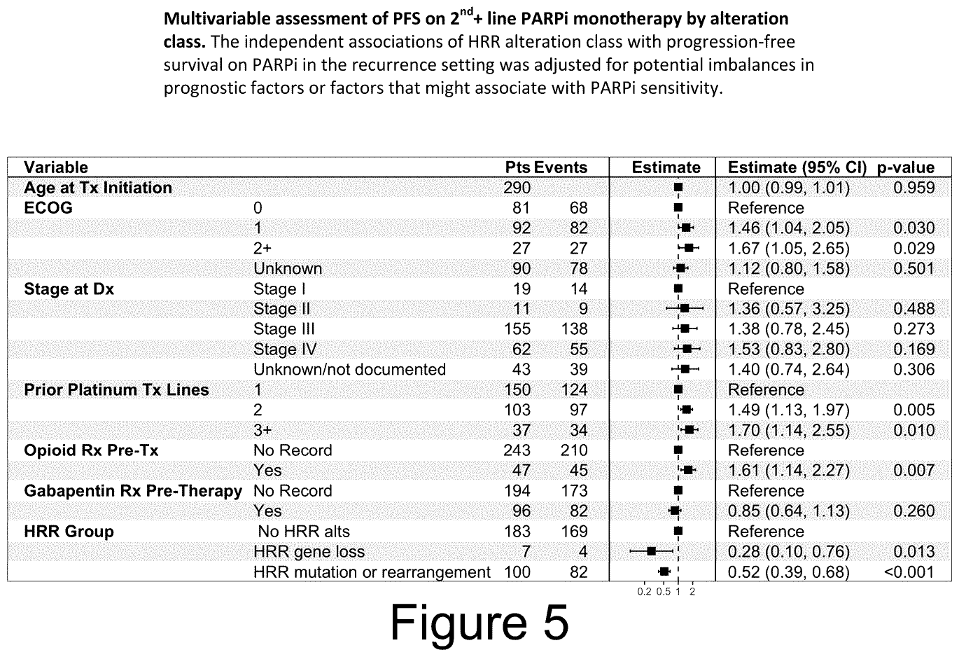

Platinum Therapy Cohort comprised patients who received platinum chemotherapy in 1st line setting and did not receive subsequent PARPi maintenance therapy, summarized in FIG. 5.

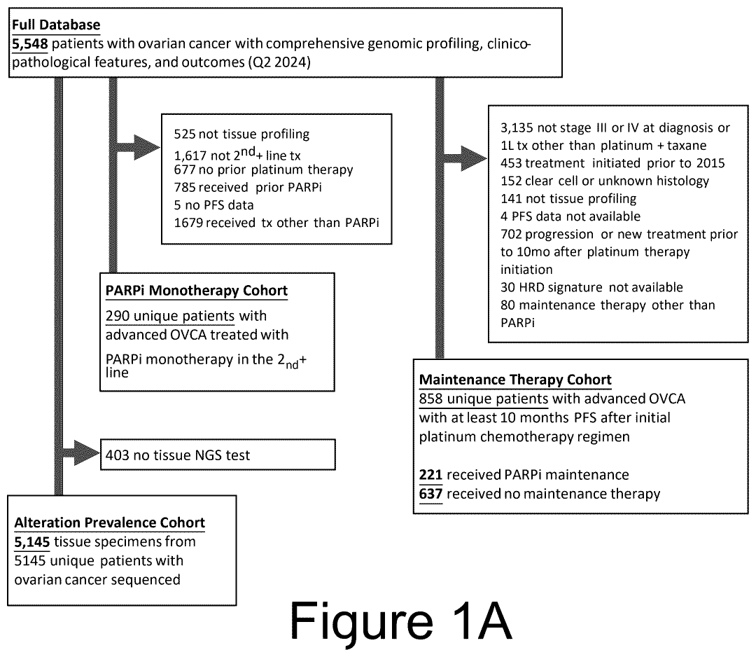

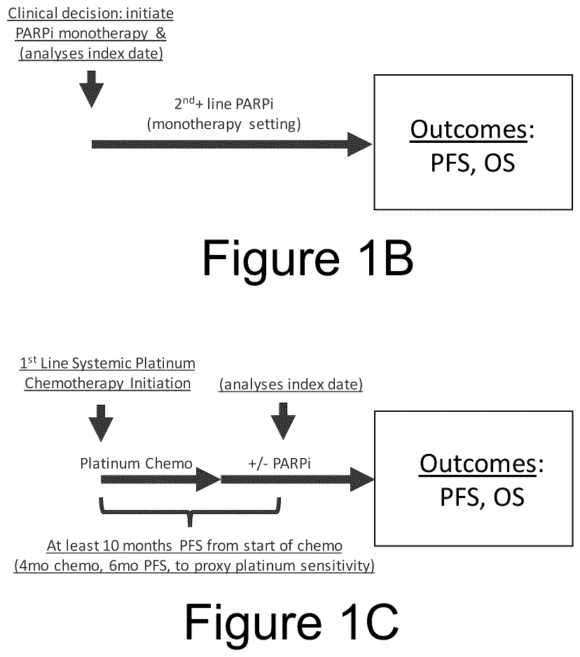

PARPi Monotherapy Cohort comprised patients who received PARPi in the 2nd or later line of therapy after progression after 1st line platinum therapy. Cohort visualization in FIG. 1.

Maintenance Therapy Cohort comprised patients who were treated with 1st line platinum chemotherapy, and did not have a progression event within 10 months after starting therapy. Direct assessment of platinum sensitivity was not available for most patients in the database, so the 10-month PFS criteria was used as a proxy for platinum sensitivity, as used previously to account for ˜4 months of chemotherapy treatment and a 6-month subsequent PFS interval. Cohort visualization in FIG. 1.

Institutional Review Board approval of the study protocol was obtained prior to study conduct and included a waiver of informed consent based on the observational, non-interventional nature of the study (WCG IRB).

VI.b. Comprehensive Genomic Profiling

Hybrid capture-based next-generation sequencing (NGS) assays were performed on patient tumor or blood specimens in a Clinical Laboratory Improvement Amendments (CLIA)-certified, College of American Pathologists (CAP)-accredited laboratory. CGP assays reporting single-nucleotide variants, insertions/deletions, genomic rearrangements, copy number amplifications, and homozygous losses.

### VII. Biomarker Definitions

Patients were considered positive for homozygous loss if they have a homozygous loss for an HRR gene, inclusive of any gene in which reversion mutations have been reported previously by a reversion mutation algorithm (REF) inclusive of: BRCA1, BRCA2, PALB2, RAD51C, RAD51D, BARD1, BRIP1, RAD51B, CDK12 via a CGP test. Patients will be considered negative if they have no such alterations on at least one CGP test. Patients will be considered positive or negative for HRR mutations or rearrangements if the same criteria are met for known/likely deleterious mutations or rearrangements.

HRDsig is a machine learning algorithm developed to predict genomic scarring consistent with homologous repair deficiency. HRDsig is a continuous factor from 0 to 1 and a cutoff of 0.7 was used.

Predominant genetic ancestry was assigned by training a random forest classifier to distinguish the 5 ancestral superpopulations of the 1000 Genomes Project10 as previously described11 determining the closest match for each specimen.

HRR gene loss status was retrospectively determined via current methods of a CGP test.

### VIII. Time to Event Outcomes

PFS was calculated from index date until the start of next treatment line (due to any cause) or death. Patients not yet reaching next treatment line or death were censored at date of last clinical visit. Real-world overall survival (OS) was calculated from index date to death from any cause, and patients with no record of mortality were right censored at the date of last clinic visit. Because patients cannot enter the database until a CGP report is delivered, OS risk intervals were left truncated to the date of CGP report to account for immortal time. Truncation independence with censoring was evaluated with Kendall's tau, with p<0.05 considered acceptable.

A database was used in which mortality information is a composite derived from 3 sources: documents within the EHR, Social Security Death Index, and a commercial death dataset mining data from obituaries and funeral homes, with validations reported in comparison to the National Death Index. The PFS measure has been benchmarked against the mortality measure, as well as assessed for reliability and repeatability. The clinco-genomic database has replicated associations with PFS and OS observed in biomarker subgroup analyses of randomized controlled trials.

### IX. Statistical Analysis

The analyses performed in this study were pre-specified in a prospectively declared statistical analysis plan (SAP), with pre-specified inclusion and exclusion criteria, potential biases, primary outcomes measures, and handling of missing data, consistent with Society for Pharmacoeconomics and Outcomes Research (ISPOR) guidelines and FDA guidance around real-world evidence (REF).

Differences in time-to-event outcomes were assessed with the log-rank test and Cox proportional hazard (PH) models. Chi-square tests and Wilcox on rank sum tests were used to assess differences between groups of categorical and continuous variables, respectively. Multiple comparison adjustments were not performed; p-values are reported to quantify the strength of association for biomarker and each outcome, not for null hypothesis significance testing, and interpretations adopted broadly considering consistency of multiple outcome measures in concert (PFS, OS) and multiple cohorts with no analysis standing on its own. OS assessments of the Maintenance Therapy Cohort were planned, but not conducted, due to only one death event among patients who received PARPi maintenance therapy and had a HRR gene loss. The SAP initially called for a propensity-weighted analysis of multiple sub-cohorts in the Maintenance Therapy Cohort, but imbalances were too great in the HRR gene loss cohort, so instead a prognostic risk score was developed using methods previously described in order to adjust for imbalances in known prognostic factors affecting time-to-event analyses.

Missing values were handled by simple imputation with expected values determined using random forests with the R package ‘missForest.’ In subsequent analyses, imputed values were treated identically to measured values. R software was used for all statistical analyses.

### X. Results

X.a. Prevalence of HRR Gene Loss

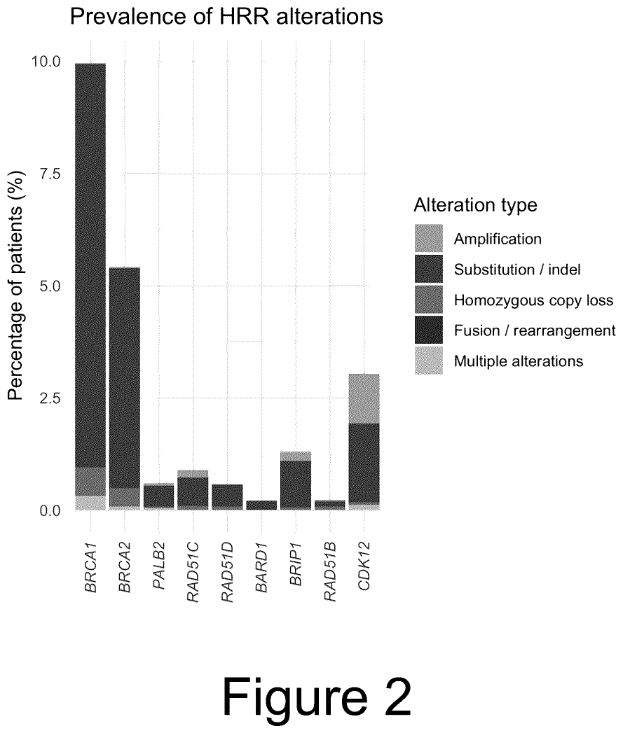

Among 5,548 patients in the overall database, 5,145 had tissue assessed with a CGP assay (FIG. 1A). Homozygous loss of HRR genes was relatively uncommon in the overall database, with the prevalence of such alterations in BRCA1, BRCA2, RAD51C, RAD51D, RAD51B, and PALB2 respectively: 0.6% (n=35), 0.5% (n=29), 0.1% (n=5), 0.1% (n=5), 0.1% (n=4) (FIG. 2).

X.b. HRR Gene Loss and PARPi Monotherapy Outcomes

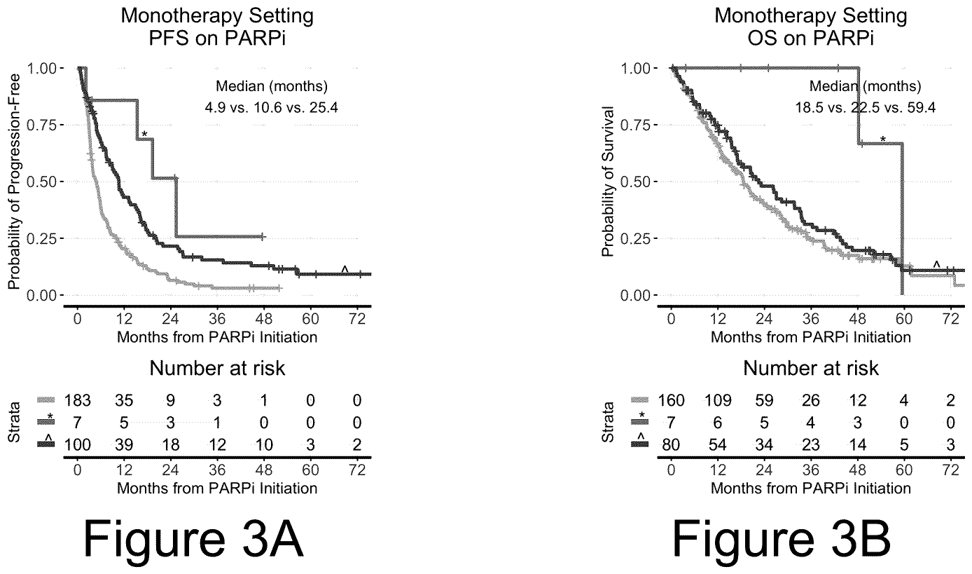

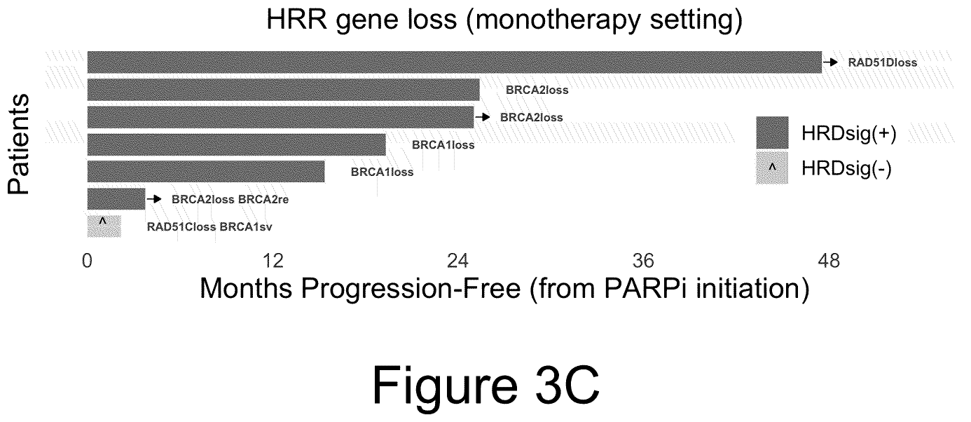

290 patients were treated with PARPi in the 2nd+ line monotherapy setting after progression on 1st line platinum+taxane chemotherapy (FIG. 1A-B). Among these, 7 had homozygous gene loss, 100 had mutations or rearrangements in HRR genes (inclusive of BRCA) and 183 did not have any detectable HRR gene alterations. The respective median PFS for these three groups were 25.4, 10.6, and 4.9 months (logrank p<0.001) (FIG. 3A). The respective OS for these three groups was 59.4, 22.5, and 18.5 months (logrank p<0.001) (FIG. 3B).

Among the 7 cases with homozygous copy loss, n=3 had BRCA2 loss, n=2 had BRCA1 loss, n=1 had RAD51D loss, and n=1 had RAD51C loss (FIG. 3C).

Patient characteristics of the PARPi Monotherapy Cohort can be found in Table 1. Multivariable analyses adjusting for pre-treatment clinical factors observed more favorable outcomes for the HRR loss (HR: 0.28, 95% CI: 0.10-0.76) and other HRR alterations groups (HR: 0.53, 95% CI: 0.39-0.68) compared to patients without HRR alterations detected for patients who received PARPi monotherapy (FIG. 5).

TABLE 1
Patient characteristics of PARPi Monotherapy Cohort

| | | | | | HRR mutation or | | | | |
|---|---|---|---|---|---|---|---|---|---|
| | No HRR alts | | HRR gene loss | | rearrangement | | Total | | |
| | (N = 183) | | (N = 7) | | (N = 100) | | (N = 290) | | p value |
| Year of 1 L Tx Initiation | | | | | | | | | <0.001 |
| 2015 | 2 | (1.1%) | 0 | (0.0%) | 6 | (6.0%) | 8 | (2.8%) | |
| 2016 | 2 | (1.1%) | 0 | (0.0%) | 8 | (8.0%) | 10 | (3.4%) | |
| 2017 | 22 | (12.0%) | 0 | (0.0%) | 19 | (19.0%) | 41 | (14.1%) | |
| 2018 | 24 | (13.1%) | 1 | (14.3%) | 13 | (13.0%) | 38 | (13.1%) | |
| 2019 | 30 | (16.4%) | 1 | (14.3%) | 20 | (20.0%) | 51 | (17.6%) | |
| 2020 | 49 | (26.8%) | 2 | (28.6%) | 13 | (13.0%) | 64 | (22.1%) | |
| 2021 | 26 | (14.2%) | 0 | (0.0%) | 2 | (2.0%) | 28 | (9.7%) | |
| 2022 | 19 | (10.4%) | 2 | (28.6%) | 9 | (9.0%) | 30 | (10.3%) | |
| 2023 | 9 | (4.9%) | 1 | (14.3%) | 10 | (10.0%) | 20 | (6.9%) | |
| Age | | | | | | | | | 0.437 |
| Median (QI, Q3) | 68.0 | (59.0, 73.5) | 72.0 | (68.0, 76.0) | 69.0 | (61.0, 73.0) | 69.0 | (60.0, 73.0) | |
| Practice Type | | | | | | | | | 0.055 |
| Academic | 31 | (17.3%) | 1 | (14.3%) | 31 | (31.3%) | 63 | (22.1%) | |
| Academic/Community | 1 | (0.6%) | 0 | (0.0%) | 2 | (2.0%) | 3 | (1.1%) | |
| Community | 147 | (82.1%) | 6 | (85.7%) | 66 | (66.7%) | 219 | (76.8%) | |
| N-Miss | 4 | | 0 | | 1 | | 5 | | |
| ECOG | | | | | | | | | 0.65 |
| 0 | 48 | (26.2%) | 3 | (42.9%) | 30 | (30.0%) | 81 | (27.9%) | |
| 1 | 60 | (32.8%) | 3 | (42.9%) | 29 | (29.0%) | 92 | (31.7%) | |
| 2+ | 18 | (9.8%) | 1 | (14.3%) | 8 | (8.0%) | 27 | (9.3%) | |
| Unknown | 57 | (31.1%) | 0 | (0.0%) | 33 | (33.0%) | 90 | (31.0%) | |
| BMI | | | | | | | | | 0.387 |
| Median (QI, Q3) | 26.5 | (21.3, 31.9) | 21.9 | (20.0, 26.7) | 25.8 | (22.7, 31.0) | 26.2 | (21.9, 31.5) | |
| N-Miss | 38 | | 0 | | 18 | | 56 | | |
| Stage at Dx | | | | | | | | | 0.759 |
| Stage I | 13 | (7.1%) | 1 | (14.3%) | 5 | (5.0%) | 19 | (6.6%) | |
| Stage II | 5 | (2.7%) | 0 | (0.0%) | 6 | (6.0%) | 11 | (3.8%) | |
| Stage III | 100 | (54.6%) | 3 | (42.9%) | 52 | (52.0%) | 155 | (53.4%) | |
| Stage IV | 41 | (22.4%) | 2 | (28.6%) | 19 | (19.0%) | 62 | (21.4%) | |
| Unknown/not | 24 | (13.1%) | 1 | (14.3%) | 18 | (18.0%) | 43 | (14.8%) | |
| documented | | | | | | | | | |
| Histology | | | | | | | | | 0.729 |
| Clear cell | 2 | (1.1%) | 0 | (0.0%) | 3 | (3.0%) | 5 | (1.7%) | |
| Epithelial NOS | 22 | (12.0%) | 1 | (14.3%) | 11 | (11.0%) | 34 | (11.7%) | |
| Serous | 147 | (80.3%) | 6 | (85.7%) | 83 | (83.0%) | 236 | (81.4%) | |
| unknown or other | 12 | (6.6%) | 0 | (0.0%) | 3 | (3.0%) | 15 | (5.2%) | |
| Opioid Rx Pre-Tx | | | | | | | | | 0.495 |
| No | 153 | (83.6%) | 7 | (100.0%) | 83 | (83.0%) | 243 | (83.8%) | |
| Yes | 30 | (16.4%) | 0 | (0.0%) | 17 | (17.0%) | 47 | (16.2%) | |
| Gabapentin Rx Pre-Therapy | | | | | | | | | 0.819 |
| No | 124 | (67.8%) | 4 | (57.1%) | 66 | (66.0%) | 194 | (66.9%) | |
| Yes | 59 | (32.2%) | 3 | (42.9%) | 34 | (34.0%) | 96 | (33.1%) | |

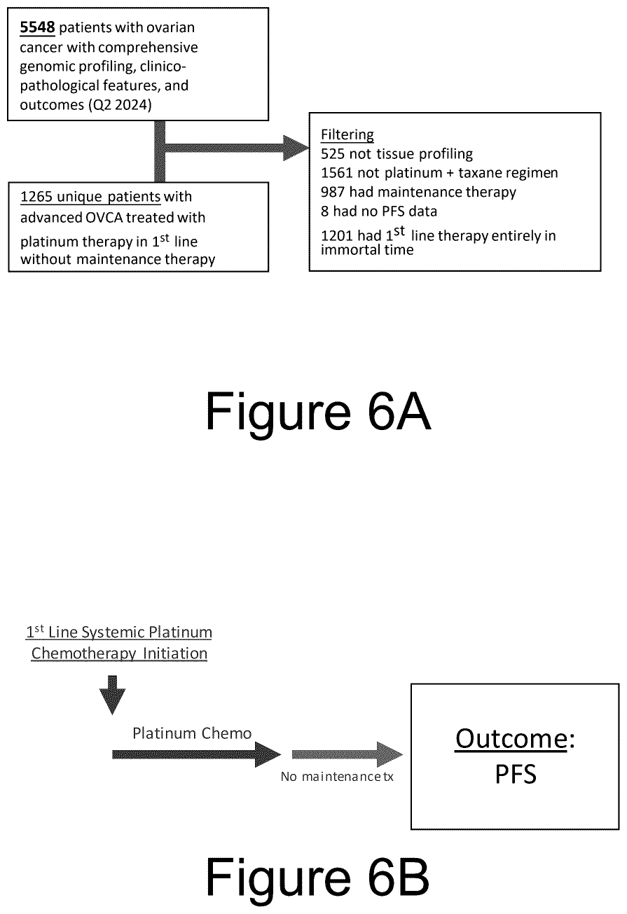

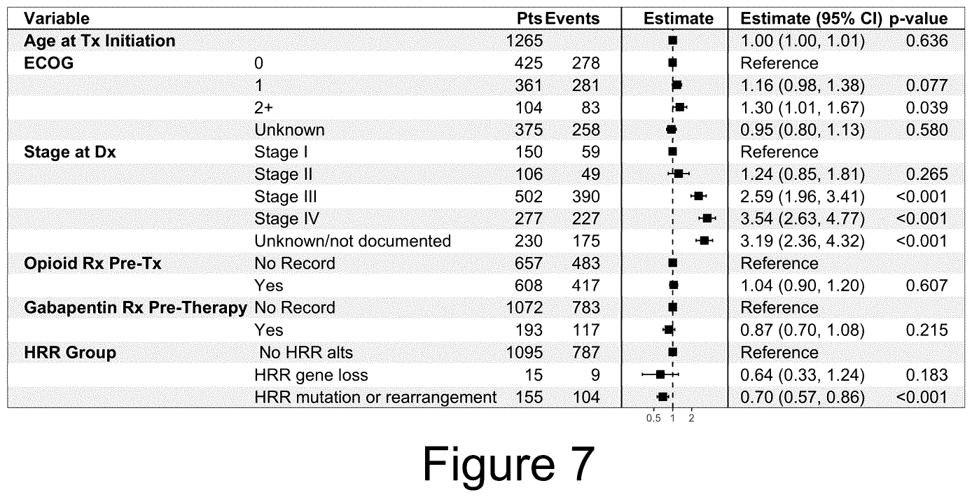

Among patients receiving 1st line platinum therapy, more favorable PFS was observed from start of platinum therapy among patients with HRR gene loss (n=15, HR: 0.64, 95% CI: 0.33-1.24) and those with other HRR alterations (n=155, HR: 0.70, 95% CI: 0.57-0.86) compared to those without HRR alterations detected (n=1095) (FIGS. 6 and 7).

X.c. HRR Gene Loss and Maintenance Therapy Outcomes

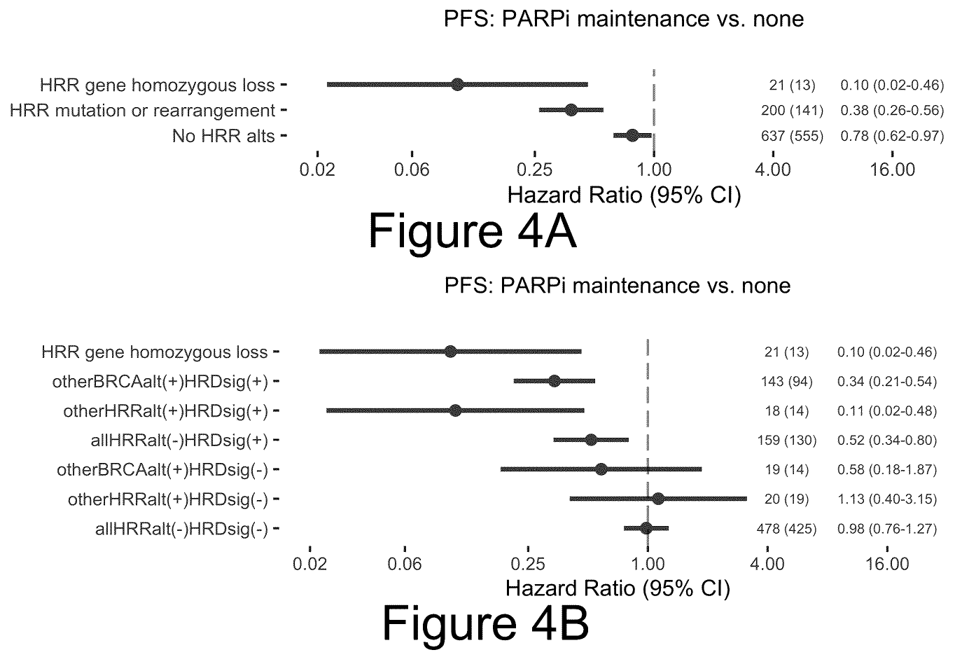

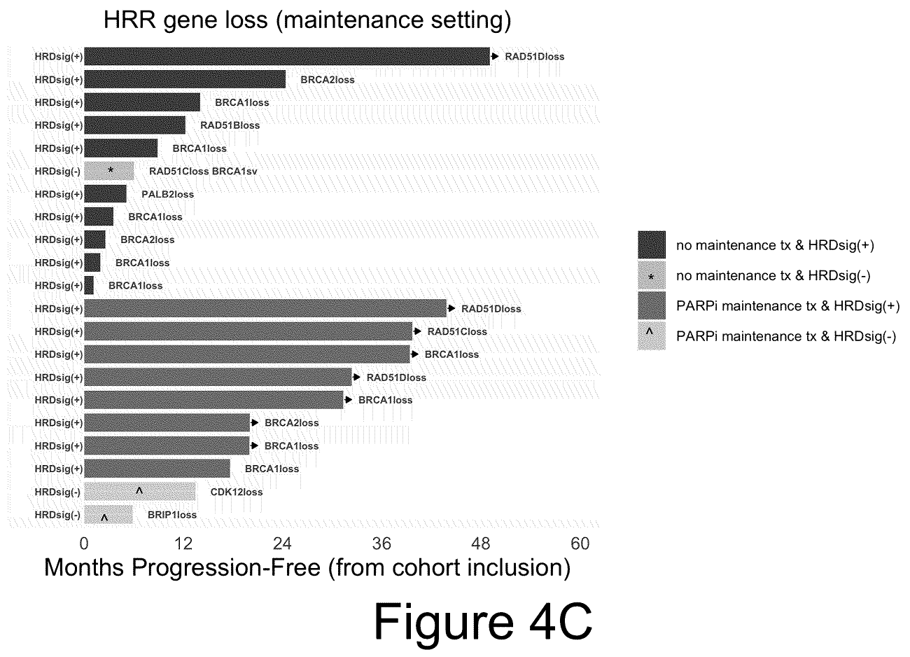

858 patients met the inclusion criteria for the Maintenance Therapy Cohort, 221 receiving PARPi maintenance therapy, and 637 receiving no maintenance therapy (FIG. 1A). The only factor associated with PARPi maintenance therapy vs. no maintenance therapy use is the year of treatment initiation (p<0.001), while other pre-treatment factors (Age, Academic vs. Community practice, ECOG performance score, body mass index, histology, opioid prescription pre-therapy, gabapentin prescription pre-therapy) were much more balanced (Table 2). Adjusting for these pre-treatment factors, PARPi maintenance therapy use was strongly associated with more favorable PFS in the presence of HRR gene homozygous loss (n=21, HR: 0.10, 95% CI: 0.02-0.46) (FIG. 4A). Presence of mutations or rearrangements in HRR genes (n=200, HR: 0.38, 95% CI: 0.26-0.56) was additionally enriched for more favorable PFS, and PAPRi use without HRR gene alterations (n=200, HR: 0.78, 95% CI: 0.62-0.97) was nominally associated with more favorable PFS as well.

TABLE 2
Patient characteristics of Maintenance Therapy Cohort

| | none (N = 637) | | PARPi (N = 221) | | Total (N = 858) | | p value |
|---|---|---|---|---|---|---|---|
| Year of 1 L Tx Initiation | | | | | | | <0.001 |
| 2015 | 116 | (18.2%) | 1 | (0.5%) | 117 | (13.6%) | |
| 2016 | 94 | (14.8%) | 1 | (0.5%) | 95 | (11.1%) | |
| 2017 | 106 | (16.6%) | 9 | (4.1%) | 115 | (13.4%) | |
| 2018 | 107 | (16.8%) | 17 | (7.7%) | 124 | (14.5%) | |
| 2019 | 84 | (13.2%) | 39 | (17.6%) | 123 | (14.3%) | |
| 2020 | 58 | (9.1%) | 56 | (25.3%) | 114 | (13.3%) | |
| 2021 | 42 | (6.6%) | 51 | (23.1%) | 93 | (10.8%) | |
| 2022 | 26 | (4.1%) | 40 | (18.1%) | 66 | (7.7%) | |
| 2023 | 4 | (0.6%) | 7 | (3.2%) | 11 | (1.3%) | |
| Age | | | | | | | 0.111 |
| Median (QI, Q3) | 66.0 | (58.0, 73.0) | 65.0 | (57.0, 71.0) | 66.0 | (58.0, 73.0) | |
| Practice Type | | | | | | | 0.682 |
| Academic | 226 | (35.5%) | 75 | (33.9%) | 301 | (35.1%) | |
| Academic/Community | 1 | (0.2%) | 1 | (0.5%) | 2 | (0.2%) | |
| Community | 410 | (64.4%) | 145 | (65.6%) | 555 | (64.7%) | |
| ECOG | | | | | | | 0.702 |
| 0 | 234 | (36.7%) | 84 | (38.0%) | 318 | (37.1%) | |
| 1 | 189 | (29.7%) | 72 | (32.6%) | 261 | (30.4%) | |
| 2+ | 50 | (7.8%) | 15 | (6.8%) | 65 | (7.6%) | |
| Unknown | 164 | (25.7%) | 50 | (22.6%) | 214 | (24.9%) | |
| BMI | | | | | | | 0.663 |
| Median (QI, Q3) | 26.9 | (22.5, 31.2) | 26.1 | (22.2, 31.7) | 26.7 | (22.4, 31.3) | |
| N-Miss | 14 | | 2 | | 16 | | |
| Stage at Dx | | | | | | | 0.024 |
| Stage III | 401 | (63.0%) | 124 | (56.1%) | 525 | (61.2%) | |
| Stage IV | 128 | (20.1%) | 64 | (29.0%) | 192 | (22.4%) | |
| Unknown/not documented | 108 | (17.0%) | 33 | (14.9%) | 141 | (16.4%) | |
| Histology | | | | | | | 0.956 |
| Epithelial NOS | 54 | (8.5%) | 19 | (8.6%) | 73 | (8.5%) | |
| Serous | 583 | (91.5%) | 202 | (91.4%) | 785 | (91.5%) | |
| Opioid Rx Pre-Tx | | | | | | | 0.255 |
| No | 371 | (58.2%) | 119 | (53.8%) | 490 | (57.1%) | |
| Yes | 266 | (41.8%) | 102 | (46.2%) | 368 | (42.9%) | |
| Gabapentin Rx Pre-Therapy | | | | | | | 0.887 |
| No | 570 | (89.5%) | 197 | (89.1%) | 767 | (89.4%) | |
| Yes | 67 | (10.5%) | 24 | (10.9%) | 91 | (10.6%) | |
| HRR Group | | | | | | | <0.001 |
| HRDsig(−) H RRalt(−) | 396 | (62.2%) | 82 | (37.1%) | 478 | (55.7%) | |
| HRDsig(−)otherBRCA(+) | 11 | (1.7%) | 8 | (3.6%) | 19 | (2.2%) | |
| HRDsig(−)otherHRR(+) | 12 | (1.9%) | 8 | (3.6%) | 20 | (2.3%) | |
| HRDsig(+) | 207 | (32.5%) | 113 | (51.1%) | 320 | (37.3%) | |
| HRR gene homozygous loss | 11 | (1.7%) | 10 | (4.5%) | 21 | (2.4%) | |

X.d. HRDsig, Non-BRCA Alterations, and Maintenance Therapy Outcomes

Among the patients who were HRDsig(+), the subset with BRCA mutations or rearrangements (n=143, HR: 0.34, 95% CI: 0.21-0.54), non-BRCA HRR alterations or rearrangements (n=18, HR: 0.11, 95% CI: 0.02-0.48), or no HRR alterations (n=159, HR: 0.52, 95% CI: 0.34-0.80) all had PFS estimates that favored PARPi maintenance therapy. However, no HRDsig(−) patient group had any substantive enrichment for more favorable PFS with PARPi maintenance therapy (FIG. 4B).

Among the 21 patients in the HRR loss group, 18 (85%) were HRDsig(+). Among the 11 patients who received maintenance PARPi, the two with HRR gene homozygous loss that were HRDsig(−) had the least favorable PFS of the group, 5.8 months (BRIP) and 13.4 months (CDK12).

### XI. Discussion

In both the maintenance and monotherapy cohorts, it was observed that the presence of HRR gene mutations or rearrangements was associated with more favorable outcomes on PARPi, but the presence of HRR gene loss had by far the most favorable PFS associations. This is also consistent with observations of patients with prostate cancer that were treated with PARPi, where the most durable benefit from PARPi was again observed among patients with homozygous loss of the HRR genes BRCA1 and BRCA2.

When groups containing BRCA or non-BRCA HRR mutations or rearrangements were further subdivided by HRDsig status, in each group, those with HRDsig(+) and PARPi maintenance therapy use were enriched for more favorable PFS, while those that were HRDsig(−) were not enriched for more favorable PFS (FIG. 4B). This is consistent with prior assessments of the database with shorter duration of follow-up6; that groups defined by HRDsig(−) status likely do not benefit from PARPi maintenance therapy.

Among the ovarian cancers in the current study with homozygous loss of HRR genes, the most common were BRCA1 and BRCA2, but homozygous loss of RAD51C and RAD51D were also associated with durable PARPi benefit (FIGS. 3C, 4C). While niraparib is approved without a biomarker for maintenance PARPi use in ovarian cancer, olaparib was approved with required companion diagnostics, which includes detection of BRCA1 or BRCA2 pathogenic mutations, rearrangements, or homozygous loss by a CGP assay utilized in the current study (REF). While most NGS tests include assessment of the BRCA1 and BRCA2 genes, it is not a given that such tests have the capability or validation necessary for the detection of BRCA homozygous loss, which requires intentional assay design and bioinformatic analysis pipelines for homozygous loss detection compared to the detection of more easily identified short variant mutations and rearrangements. To our knowledge, completed clinical trials of PARPi use in ovarian cancer have not idistinguished the outcome associations of HRR gene loss detection in contrast with other short variant inactivating mutations in HRR genes. Finally, the enhanced durability of response to PARPi-based treatments in advanced ovarian cancer in this study among patients whose tumors feature HRR gene homozygous loss rather than HRR gene inactivating short variant mutations could be further validated by subgroup analyses of existing, completed clinical trials.

### XII. Limitations

Observational and/or retrospective analyses are more prone to false discovery than prospective randomized trials, due to multiple hypothesis testing and potential imbalances between groups. Use of a prospectively declared statistical analysis plan was made to reduce the risk of false discovery, and rigorous adjustment of prognostic factors was performed to reduce potential imbalances between groups that might confound time-to-event comparisons. However, these adjustments do not account for all potential imbalances. Clinical annotations of NGS tests, bioinformatic pipelines, and reporting can vary between laboratories. Therefore, the results from this study are not generalizable to biomarker performance of all NGS platforms.

### XIII. Conclusions

In routine practice settings, the identification of homozygous loss of HRR genes in advanced ovarian cancer patients is rare (˜1.5%) but this specific type of HRR gene alteration is associated with the most durable benefit from PARPi. NGS tests that are validated to detect homozygous loss of HRR genes should be prioritized for clinical use to guide PARPi treatment decisions.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLE CLAUSES

The following clauses provide various examples of implementations of the present disclosure. However, the scope of the disclosure is not limited to any of the clauses listed below.
1. A method, including: providing a plurality of nucleic acid molecules obtained from a sample from a subject with ovarian cancer; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data, the sequence read data being in a spatial domain corresponding to genomic position within at least one locus of a genome of the sample; receiving, at one or more processors, the sequence read data for the plurality of sequence reads; determining, by the one or more processors and based on the sequence read data, that the subject has homozygous loss of a homologous recombination repair (HRR) gene; and predicting, by the one or more processors and based on the homozygous loss of the HRR gene, that a treatment including a poly (ADP-ribose) polymerase (PARP) inhibitor will treat the ovarian cancer of the subject.
2. The method of clause 1, wherein determining, by the one or more processors and based on the sequence read data, that the subject has the homozygous loss of the HRR gene includes: determining that the sequence read data indicates loss of at least 2,000 base pairs of each copy of the HRR gene of the subject.
3. The method of clause 1 or 2, wherein determining, by the one or more processors and based on the sequence read data, that the subject has the homozygous loss of the HRR gene includes:
determining that the sequence read data indicates loss of at least 10% of base pairs of each copy of the HRR gene of the subject.
4. The method of any of clauses 1 to 3, wherein the HRR gene encodes RAD51B, RAD51C, or RAD51E.
5. The method of any of clauses 1 to 4, further including: determining, by the one or more processors and based on the sequence read data, that the subject has HRD, wherein predicting that the PARP inhibitor will treat the ovarian cancer of the subject is further based on determining that the subject has homologous recombination deficiency (HRD).
6. The method of any of clauses 1 to 5, further including: administering, to the subject, a platinum-containing chemotherapy; and administering, to the subject, the treatment including the PARP inhibitor.
7. A method, including: identifying sequence read data indicating sequences of one or more nucleic acid molecules of a sample obtained from a subject with cancer; determining, based on the sequence read data, that the subject has homozygous loss of an HRR gene; and predicting that the cancer is susceptible to a treatment including a PARP inhibitor based on the homozygous loss of the HRR gene.
8. The method of clause 7, wherein the sample includes a tissue biopsy sample or a liquid biopsy sample.
9. The method of clause 7 or 8, wherein the sample includes a liquid biopsy sample.
10. The method of clause 9, wherein the liquid biopsy sample includes blood, plasma, cerebrospinal fluid, sputum, stool, urine, lymphatic fluid, or saliva.
11. The method of any of clauses 9 to 10, wherein the liquid biopsy sample includes circulating tumor cells (CTCs).
12. The method of any of clauses 7 to 11, wherein the sample includes a blood sample.
13. The method of any of clauses 7 to 12, wherein the sample includes plasma.
14. The method of any of clauses 7 to 13, wherein the sample includes a tissue biopsy sample.
15. The method of clause 14, wherein the tissue biopsy sample is obtained from a tumor of the subject.
16. The method of clause 15, wherein the tumor includes a primary tumor.
17. The method of clause 15 or 16, wherein the tumor includes a secondary tumor.
18. The method of any of clauses 14 to 17, wherein the tissue biopsy sample includes a tissue of an organ and/or differentiated tissue of the subject.
19. The method of any of clauses 7 to 18, wherein the sample includes cell-free DNA (cfDNA) and/or genomic DNA.
20. The method of clause 19, wherein the cfDNA includes ctDNA.
21. The method of any of clauses 7 to 20, further including: receiving the sample.
22. The method of any of clauses 7 to 21, further including: extracting the one or more nucleic acid molecules from the sample.
23. The method of any of clauses 7 to 22, wherein the one or more nucleic acid molecules include genomic DNA.
24. The method of any of clauses 7 to 23, further including: ligating one or more adapters onto the one or more nucleic acid molecules in the sample; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, wherein the sequence read data is indicative of the sequence reads, thereby generating the sequence read data.
25. The method of clause 24, wherein the one or more adapters include at least one of amplification primers, flow cell adaptor sequences, substrate adapter sequences, or sample index sequences.
26. The method of clause 24 or 25, wherein the captured nucleic acid molecules are captured from the amplified nucleic acid molecules by hybridization to one or more bait molecules.
27. The method of clause 26, wherein the one or more bait molecules include one or more additional nucleic acid molecules, each of the one or more additional nucleic acid molecules including a region that is complementary to a region of a captured nucleic acid molecule.
28. The method of any of clauses 24 to 27, wherein amplifying the one or more ligated nucleic acid molecules includes performing a polymerase chain reaction (PCR) amplification technique, a non-PCR amplification technique, or an isothermal amplification technique.
29. The method of any of clauses 24 to 28, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
30. The method of any of clauses 24 to 29, wherein sequencing the captured nucleic acid molecules includes next generation sequencing (NGS).
31. The method of any of clauses 24 to 30, wherein sequencing the captured nucleic acid molecules is performed by a next generation sequencer.
32. The method of any of clauses 24 to 31, wherein sequencing the captured nucleic acid molecules includes sequencing-by-synthesis or nanopore sequencing.
33. The method of any of clauses 7 to 32, further including: generating ligated molecules by ligating adaptors onto the one or more nucleic acid molecules of the sample; generating amplified ligated molecules by amplifying the ligated molecules; generating, using the amplified ligated molecules, detection signals; detecting, by at least one sensor, the detection signals; and generating the sequence read data based on the detection signals.
34. The method of clause 33, wherein the detection signals include electrical signals and/or optical signals.
35. The method of clause 34, wherein generating, using the amplified ligated molecules, the detection signals includes simultaneously: synthesizing, by a polymerase using fluorescently tagged nucleotide triphosphates (NTPs), a synthesized nucleic acid molecule based on one of the amplified ligated molecules, and wherein detecting, by the at least one sensor, the detection signals include: detecting, by at least one optical sensor, optical signals emitted by the fluorescently tagged NTPs upon binding to the synthesized nucleic acid molecule, the optical signals being indicative of at least one sequence of one or more nucleic acid molecules.
36. The method of clause 34 or 35, wherein generating, using the amplified ligated molecules, the detection signals include simultaneously: directing the amplified ligated molecules through a nanopore extending from a first space to a second space through a substrate, and wherein detecting, by the at least one sensor, the detection signals include: detecting, by sensors disposed in the first space and the second space, an electrical signal over time, the electrical signal being indicative of at least one sequence of the one or more nucleic acid molecules.
37. The method of any of clauses 7 to 36, wherein the subject is human.
38. The method of any of clauses 7 to 37, wherein the subject has ovarian cancer.
39. The method of any of clauses 7 to 38, wherein the subject has prostate cancer.
40. The method of any of clauses 7 to 39, wherein the subject has breast cancer.
41. The method of any of clauses 7 to 40, wherein the subject has pancreatic cancer.
42. The method of any of clauses 7 to 41, wherein the subject has colorectal cancer, esophagogastric cancer, hepatobiliary cancer, a melanoma, non-melanoma skin cancer, lung cancer, kidney cancer, endometrial cancer, or bladder cancer.
43. The method of any of clauses 7 to 42, wherein the sequence read data corresponds to at least one genomic locus.
44. The method of clause 43, wherein the at least one genomic locus includes the HRR gene.
45. The method of any of clauses 7 to 44, wherein the HRR gene encodes RAD51B, RAD51C, or RAD51D.
46. The method of any of clauses 7 to 45, wherein the HRR gene encodes RAD51B.
47. The method of any of clauses 7 to 46, wherein the HRR gene encodes RAD51C.
48. The method of any of clauses 7 to 47, wherein the HRR gene encodes RAD51D.
49. The method of any of clauses 7 to 48, wherein the HRR gene encodes PALB2, BARD1, BRIP1, or CDK12.
50. The method of any of clauses 7 to 49, wherein the HRR gene encodes BRCA1 or BRCA2.
51. The method of any of clauses 7 to 50, wherein determining, based on the sequence read data, that the subject has homozygous loss of the HRR gene includes:
determining that the sequence read data indicates that the subject has a loss of each copy of the HRR gene.
52. The method of any of clauses 7 to 51, wherein determining, based on the sequence read data, that the subject has homozygous loss of the HRR gene includes:
determining, based on the sequence read data, that each copy of the HRR gene of the subject omits greater than a threshold amount of base pairs compared to the HRR gene in a reference genome.
53. The method of clause 52, wherein the threshold amount includes 1,000 base pairs.
54. The method of clause 52 or 53, wherein the threshold amount includes 2,000 base pairs.
55. The method of any of clauses 52 to 54, wherein the threshold amount includes 10% of a total number of base pairs of the HRR gene in the reference genome.
56. The method of any of clauses 52 to 55, wherein the threshold amount includes 20% of a total number of base pairs of the HRR gene in the reference genome.
57. The method of any of clauses 7 to 56, wherein the PARP inhibitor includes at least one of olaparib, niraparib, rucaparib, or talazoparib.
58. The method of any of clauses 7 to 57, further including: determining, based on the sequence read data, a mutational profile of the sample; inputting the mutational profile into a model, wherein the model is trained using training data related to a plurality of mutational signatures; and predicting one or more mutational signatures of the plurality of mutational signatures associated with the sample based on an output of the model, wherein the output of the model is associated with a dimensionality value that is less than a number of the plurality of mutational signatures, and wherein predicting that the cancer is susceptible to the treatment including a PARP inhibitor is further based on the one or more mutational signatures.
59. The method of clause 58, wherein the model includes an autoencoder model.
60. The method of any of clauses 7 to 59, further including: determining, based on the sequence read data, a mismatch repair deficiency (MMRD) probability score, the MMRD probability score being indicative of a functional deficiency in at least one MMR gene including the MMR gene, wherein predicting that the cancer is susceptible to the treatment including the PARP inhibitor is further based on the MMRD probability score.
61. The method of clause 60, wherein determining, based on the sequence read data, the MMRD probability score includes: generating, by extracting two or more features of the sequence read data, input features; and inputting the input features into a predictive model configured to generate the MMRD probability score based on the input features.
62. The method of clause 60 or 61, wherein predicting that the cancer is susceptible to the treatment including the PARP inhibitor is based on determining that the MMRD probability score is above a threshold.
63. The method of any of clauses 60 to 62, further including: determining, based on the MMRD probability score, that the subject is MMR deficient, wherein predicting that the cancer is susceptible to the treatment including the PARP inhibitor is based on determining that the subject is MMR deficient.
64. The method of any of clauses 7 to 63, further including: determining, based on the sequence read data, that the subject has HRD, wherein predicting that the cancer is susceptible to the treatment including the PARP inhibitor is further based on determining that the subject has HRD.
65. The method of clause 64, wherein determining, based on the sequence read data, that the subject has HRD includes: determining, based on the sequence read data, a genomic instability score of the subject indicating at least one of a: loss of heterozygosity in a genome of the subject; telomeric allelic imbalance of the subject; or large-scale state transitions in the genome of the subject; and comparing the genomic instability score to a threshold.
66. The method of clause 64 or 65, wherein determining, based on the sequence read data, that the subject has HRD includes: determining, based on the sequence read data, a metric indicative of at least one of: microsatellite instability of the subject; a tumor mutational burden of the subject; or one or more variants in one or more genes of the subject; and comparing the metric to a threshold.
67. The method of any of clauses 64 to 66, wherein determining, based on the sequence read data, that the subject has HRD includes: inputting features of the sequence read data into a trained homologous recombination deficiency (HRD) model; and classifying, using the trained HRD model, a tumor of the subject as HRD-positive.
68. The method of clause 67, wherein the features of the sequence read data include features of genomic regions that omit the HRR gene.
69. The method of any of clauses 7 to 68, further including: generating, based on the homozygous loss and/or predicting that the cancer is susceptible to the treatment including the PARP inhibitor, a genomic profile of the subject.
70. The method of clause 69, wherein the genomic profile includes results from at least one of: a histological study, whole transcriptome sequencing, cfRNA sequencing, a comprehensive genomic profiling test; a whole genome sequencing (WGS) test; a whole exome sequencing (WES) test; a gene expression profiling test; a cancer hotspot panel test; a DNA methylation test; a DNA fragmentation test; or an RNA fragmentation test, a microsatellite instability (MSI) test, a tumor mutational burden (TMB) test, or a viral status test.
71. The method of clause 69 or 70, wherein the genomic profile of the subject includes: results from a nucleic acid sequencing-based test.
72. The method of any of clauses 7 to 71, further including: based on predicting that the cancer is susceptible to the treatment including the PARP inhibitor, generating the treatment for the subject.
73. The method of clause 72, wherein the treatment includes drug therapy, radiation therapy, a targeted therapy, vaccine therapy, stem cell transplantation, blood transfusion, physical therapy, psychiatric therapy, or surgery.
74. The method of clause 73, wherein the drug therapy includes chemotherapy.
75. The method of clause 74, wherein the chemotherapy includes a platinum-containing chemotherapy.
76. The method of any of clauses 73 to 75, wherein the targeted therapy includes immunotherapy or genetic therapy.
77. The method of clause 76, wherein the targeted therapy includes bevacizumab.
78. The method of any of clauses 7 to 77, further including: administering the treatment to the subject.
79. The method of any of clauses 7 to 78, further including determining, based on the homozygous loss of the HRR gene and/or predicting that the cancer is susceptible to the treatment including the PARP inhibitor, whether to perform a follow-up diagnostic test.
80. The method of clause 79, further including performing the follow-up diagnostic test.
81. The method of clause 79 or 80, wherein the follow-up diagnostic test includes a physical exam, biopsy, sequence-based test, diagnostic imaging, histological study, or viral status test.
82. The method of clause 81, wherein the biopsy includes obtaining a tissue biopsy sample of a tumor of the subject.
83. The method of clause 81 or 82, wherein the sequence-based test includes whole transcriptome sequencing, cfRNA sequencing, whole exome sequencing, whole genome sequencing, a cancer hotspot panel test, a DNA methylation test, a DNA fragmentation test, an RNA fragmentation test, a microsatellite instability (MSI) test, or a tumor mutational burden (TMB) test.
84. The method of any of clauses 81 to 83, wherein the diagnostic imaging includes magnetic resonance imaging, computed tomography scan, ultrasound, X-ray, mammogram, positron emission tomography, bone scintigraphy, myelography, virtual colonoscopy, echocardiography, radiography, nuclear medicine, fluoroscopy, or single-photon emission computed tomography.
85. The method of any of clauses 80 to 84, wherein the follow-up diagnostic test includes at least one of: whole transcriptome sequencing; cfRNA sequencing; or an RNA fragmentation test.
86. The method of any of clauses 7 to 85, further including determining, based on the homozygous loss of the HRR gene and/or predicting that the cancer is susceptible to the treatment including the PARP inhibitor, whether the subject is eligible for a clinical trial.
87. The method of clause 86, wherein determining, based on the homozygous loss of the HRR gene and/or predicting that the cancer is susceptible to the treatment including the PARP inhibitor, whether the subject is eligible for the clinical trial includes determining that the subject matches inclusion criteria for the clinical trial.
88. The method of clause 87, wherein the inclusion criteria include criteria for age, gender, disease stage, and previous treatments.
89. The method of any of clauses 86 to 88, wherein determining, based on the homozygous loss of the HRR gene and/or predicting that the cancer is susceptible to the treatment including the PARP inhibitor, whether the subject is eligible for the clinical trial includes determining that the subject is taking one or more specific medications.
90. The method of any of clauses 86 to 89, wherein determining, based on the homozygous loss of the HRR gene and/or predicting that the cancer is susceptible to the treatment including the PARP inhibitor, whether the subject is eligible for the clinical trial includes determining that the subject is not taking any medications.
91. The method of any of clauses 86 to 90, wherein the subject is not eligible for the clinical trial.
92. The method of any of clauses 7 to 91, further including: generating a report based on the homozygous loss of the HRR gene and/or predicting that the cancer is susceptible to the treatment including the PARP inhibitor; and outputting the report.
93. The method of clause 92, wherein outputting the report includes: transmitting data indicating the report to an external device.
94. The method of clause 93, wherein the external device is associated with the subject and/or a healthcare provider.
95. The method of clause 93 or 94, wherein the data is transmitted over one or more communication networks.
96. The method of any of clauses 93 to 95, wherein the data is transmitted over a peer-to-peer connection.
97. The method of any of clauses 93 to 96, wherein outputting the report includes: visually presenting, by a display, the report.
98. The method of any of clauses 93 to 97, wherein the report indicates the homozygous loss of the HRR gene and/or an indication that the cancer is predicted to be susceptible to the treatment including the PARP inhibitor.
99. A system, including: at least one processor; and memory storing instructions that, when executed by the at least one processor, cause the at least one processor to perform operations including: identifying sequence read data indicating sequences of one or more nucleic acid molecules of a sample obtained from a subject with cancer; determining, based on the sequence read data, that the subject has homozygous loss of an HRR gene; and predicting that the cancer is susceptible to a treatment including a PARP inhibitor based on the homozygous loss of the HRR gene.
100. The system of clause 99, further including: a sequencer configured to generate the sequence read data by sequencing the one or more nucleic acid molecules in the sample.
101. The system of clause 99 or 100, further including: a transceiver configured to transmit data indicating the homozygous loss of the HRR gene and/or that the cancer is predicted to be susceptible to the treatment including the PARP inhibitor.
102. The system of any of clauses 99 to 101, further including: an output device configured to output an indication of the homozygous loss of the HRR gene and/or that the cancer is predicted to be susceptible to the treatment including the PARP inhibitor.
103. A non-transitory computer readable medium storing instructions for performing operations including: identifying sequence read data indicating sequences of one or more nucleic acid molecules of a sample obtained from a subject with cancer; determining, based on the sequence read data, that the subject has homozygous loss of an HRR gene; and predicting that the cancer is susceptible to a treatment including a PARP inhibitor based on the homozygous loss of the HRR gene.
104. A method of identifying a subject having a cancer susceptible to a treatment including a PARP inhibitor, the method including detecting in a sample from the subject: homozygous loss of an HRR gene, wherein detection of the homozygous loss of the HRR gene identifies the subject as one who has the cancer susceptible to the treatment including the PARP inhibitor.
105. The method of clause 104, wherein the HRR gene encodes RAD51B, RAD51C, or RAD51E.
106. The method of clause 104 or 105, wherein the cancer includes ovarian cancer.
107. A method of treating or delaying progression of a cancer in a subject in need thereof, including: acquiring knowledge of a homozygous loss of an HRR gene in one or more nucleic acid molecules obtained from a sample of the subject; selecting a treatment including a PARP inhibitor based on the acquired knowledge; and administering to the subject an effective amount of the treatment.
108. The method of clause 107, wherein the HRR gene encodes RAD51B, RAD51C, or RAD51E.
109. The method of clause 107 or 108, wherein the cancer includes ovarian cancer.
110. The method of any of clauses 107 to 109, further including: acquiring knowledge of an HRD status of the subject, wherein selecting the treatment including the PARP inhibitor is further based on the knowledge of the HRD status of the subject.
111. A method of treating a subject having ovarian cancer, the method including: acquiring a genomic profile based on a sample from the subject, wherein the genomic profile is indicative of: (i) an HRDsig positive status, and (ii) the presence of a RAD51C loss or a RAD51D loss; and responsive to an indication that an HRD positive status is present with the presence of at least one of a RAD51C loss or a RAD51D loss, administering an anti-cancer therapy to the subject, thereby treating the subject with the anti-cancer therapy.
112. The method of clause 111, wherein the anti-cancer therapy includes a PARP inhibitor.
113. A method for identifying a cancer patient as a candidate for treatment with a PARP inhibitor, the method including: receiving, at one or more processors, genomic data derived from a sample from the subject; providing, using the one or more processors, the genomic data as input to a first trained model configured to identify a HRDsig status; providing, using the one or more processors, the genomic data as input to a second trained machine model configured to identify a RAD51C status and/or a RAD51D status; and identifying the cancer patient as a candidate for treatment with the PARP inhibitor based on: (i) an output from the first trained model is indicative that the HRDsig status is positive, and (ii) an output from the second trained model is indicative of a RAD51C loss and/or a RAD51D loss.
114. The method of clause 113, wherein the first trained model and the second trained model are a singular model 115. The method of clause 113 or 114, wherein the cancer includes an ovarian cancer.

1. A method, comprising:
providing a plurality of nucleic acid molecules obtained from a sample from a subject with ovarian cancer;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data, the sequence read data being in a spatial domain corresponding to genomic position within at least one locus of a genome of the sample;
receiving, at one or more processors, the sequence read data for the plurality of sequence reads;
determining, by the one or more processors and based on the sequence read data, that the subject has homozygous loss of a homologous recombination repair (HRR) gene; and
predicting, by the one or more processors and based on the homozygous loss of the HRR gene, that a treatment comprising a poly (ADP-ribose) polymerase (PARP) inhibitor will treat the ovarian cancer of the subject.

2-5. (canceled)

6. The method of claim 1, further comprising:
administering, to the subject, a platinum-containing chemotherapy; and
administering, to the subject, the treatment comprising the PARP inhibitor.

7. A method, comprising:
identifying sequence read data indicating sequences of one or more nucleic acid molecules of a sample obtained from a subject with cancer;
determining, based on the sequence read data, that the subject has homozygous loss of an HRR gene; and
predicting that the cancer is susceptible to a treatment comprising a PARP inhibitor based on the homozygous loss of the HRR gene.

8. The method of claim 7, wherein the sample comprises a tissue biopsy sample or a liquid biopsy sample.

9-41. (canceled)

42. The method of claim 7, wherein the subject has ovarian cancer, prostate cancer, breast cancer, pancreatic cancer, colorectal cancer, esophagogastric cancer, hepatobiliary cancer, a melanoma, non-melanoma skin cancer, lung cancer, kidney cancer, endometrial cancer, or bladder cancer.

43. The method of claim 7, wherein the sequence read data corresponds to at least one genomic locus.

44. (canceled)

45. The method of claim 7, wherein the HRR gene encodes RAD51B, RAD51C, RAD51D, PALB2, BARD1, BRIP1, CDK12, BRCA1, or BRCA2.

46-50. (canceled)

51. The method of claim 7, wherein determining, based on the sequence read data, that the subject has homozygous loss of the HRR gene comprises:
determining that the sequence read data indicates that the subject has a loss of each copy of the HRR gene; or
determining, based on the sequence read data, that each copy of the HRR gene of the subject omits greater than a threshold amount of base pairs compared to the HRR gene in a reference genome.

52. (canceled)

53. The method of claim 51, wherein the threshold amount comprises 1,000 base pairs or 2,000 base pairs; or
wherein the threshold amount comprises 10% of a total number of base pairs of the HRR gene in the reference genome or 20% of the total number of base pairs of the HRR gene in the reference genome.

54-57. (canceled)

58. The method of claim 7, further comprising:
determining, based on the sequence read data, a mutational profile of the sample;
inputting the mutational profile into a model, wherein the model is trained using training data related to a plurality of mutational signatures; and
predicting one or more mutational signatures of the plurality of mutational signatures associated with the sample based on an output of the model,
wherein the output of the model is associated with a dimensionality value that is less than a number of the plurality of mutational signatures, and
wherein predicting that the cancer is susceptible to the treatment including a PARP inhibitor is further based on the one or more mutational signatures.

59. (canceled)

60. The method of claim 7, further comprising:
determining, based on the sequence read data, a mismatch repair deficiency (MMRD) probability score, the MMRD probability score being indicative of a functional deficiency in at least one MMR gene comprising the MMR gene,
wherein predicting that the cancer is susceptible to the treatment including the PARP inhibitor is further based on the MMRD probability score.

61-63. (canceled)

64. The method of claim 7, further comprising:
determining, based on the sequence read data, that the subject has homologous recombination deficiency (HRD),
wherein predicting that the cancer is susceptible to the treatment including the PARP inhibitor is further based on determining that the subject has HRD.

65-71. (canceled)

72. The method of claim 7, further comprising:
based on predicting that the cancer is susceptible to the treatment comprising the PARP inhibitor, generating the treatment for the subject.

73. The method of claim 72, wherein the treatment comprises drug therapy, radiation therapy, a targeted therapy, vaccine therapy, stem cell transplantation, blood transfusion, physical therapy, psychiatric therapy, or surgery.

74. The method of claim 73, wherein the drug therapy comprises a platinum-containing chemotherapy; and/or
wherein the targeted therapy comprises immunotherapy or genetic therapy.

75-77. (canceled)

78. The method of claim 7, further comprising:
administering the treatment to the subject.

79-85. (canceled)

86. The method of claim 7, further comprising determining, based on the homozygous loss of the HRR gene and/or predicting that the cancer is susceptible to the treatment comprising the PARP inhibitor, whether the subject is eligible for a clinical trial.

87-91. (canceled)

92. The method of claim 7, further comprising:
generating a report based on the homozygous loss of the HRR gene and/or predicting that the cancer is susceptible to the treatment comprising the PARP inhibitor; and
outputting the report.

93-98. (canceled)

99. A system, comprising:
at least one processor; and
memory storing instructions that, when executed by the at least one processor, cause the at least one processor to perform operations comprising:
identifying sequence read data indicating sequences of one or more nucleic acid molecules of a sample obtained from a subject with cancer;
determining, based on the sequence read data, that the subject has homozygous loss of an HRR gene; and
predicting that the cancer is susceptible to a treatment comprising a PARP inhibitor based on the homozygous loss of the HRR gene.

100. (canceled)

101. The system of claim 99, further comprising:
a transceiver configured to transmit data indicating the homozygous loss of the HRR gene and/or that the cancer is predicted to be susceptible to the treatment comprising the PARP inhibitor.

102-115. (canceled)
